(19) United States
(12) Patent Application Publication
(10) Pub. No.: US 2005/0216092 A1
(43) Pub. Date: Sep. 29, 2005

(54) Constrained artificial implant for orthopaedic applications

(75) Inventors: Greg Marik, Germantown, TN (US); Kevin Foley, Germantown, TN (US); Tom Francis, Cordova, TN (US); Randall Allard, Germantown, TN (US)

Correspondence Address: HAYNES AND BOONE, LLP, 901 MAIN ST, SUITE 3100, DALLAS, TX, 75202, US

(73) Assignee: SDGI Holdings, inc., Wilmington, DE (US)

(21) Appl. No.: 10/806,487

(22) Filed: Mar. 23, 2004

(51) Int. Cl. A61F 2/30; A61F 2/44

(52) U.S. Cl. 623/23.39; 623/17.14; 623/17.15

(57) ABSTRACT

A joint prosthesis comprises a first member for engaging a first bone portion and a second member for engaging a second bone portion. The first member comprises a first surface with a first curve and the second member comprises a second surface with a second curve. The first member is translatable with respect to the second member and the second curve is positioned within the first curve to bias the first and second curves towards alignment along a first axis passing through the first and second bone portions.

### BACKGROUND

During the past thirty years, technical advances in the design of large joint reconstructive devices has revolutionized the treatment of degenerative joint disease, moving the standard of care from arthrodesis to arthroplasty. Reconstruction of a damaged joint with a functional joint prosthesis to provide motion and to reduce deterioration of the adjacent bone and adjacent joints is a desirable treatment option for many patients. Current prosthesis designs, however, may not provide the stability needed to achieve the desired results.

### SUMMARY

In one embodiment, a joint prosthesis comprises a first member for engaging a first bone portion and a second member for engaging a second bone portion. The first member comprises a first surface with a first curve, and the second member comprises a second surface with a second curve. The first member is translatable with respect to the second member and the second curve is positioned within the first curve to bias the first and second curves towards alignment along a first axis passing through the first and second bone portions.

### BRIEF DESCRIPTION OF THE DRAWINGS

FIG. 1 is a human anatomy.

FIG. 2 is a block drawing of a human joint.

FIG. 3 is a sagittal view of a vertebral column having a damaged disc.

FIG. 4 is an exploded intervertebral assembly according to a first embodiment of the current disclosure.

FIG. 5 is an assembled intervertebral assembly according to the first embodiment of the current disclosure.

FIG. 6 is a sagittal view of a vertebral column implanted with the intervertebral assembly according to the first embodiment of the current disclosure.

FIG. 7 is a cross sectional view of the assembled intervertebral assembly according to the first embodiment of the current disclosure.

FIG. 8 is a cross sectional view of the translated intervertebral assembly according to the first embodiment of the current disclosure.

FIG. 9 is a cross sectional view of an assembled intervertebral assembly according to a second embodiment of the current disclosure.

FIG. 10 is a cross sectional view of an assembled intervertebral assembly according to a third embodiment of the current disclosure.

FIG. 11 is a cross sectional view of an assembled intervertebral assembly according to a fourth embodiment of the current disclosure.

FIG. 12 is a cross sectional view of an assembled intervertebral assembly according to a fifth embodiment of the current disclosure.

FIG. 13 is a cross sectional view of an assembled intervertebral assembly according to a sixth embodiment of the current disclosure.

FIG. 14 is a cross sectional view of an assembled intervertebral assembly according to a seventh embodiment of the current disclosure.

FIG. 15 is an exploded intervertebral assembly according to an eighth embodiment of the current disclosure.

FIG. 16 is an assembled intervertebral assembly according to the eighth embodiment of the current disclosure.

FIG. 17 is a cross sectional view of the assembled intervertebral assembly of the eighth embodiment of the current disclosure in a translated position.

FIG. 18 is an exploded intervertebral assembly according to a ninth embodiment of the current disclosure.

FIG. 19 is an assembled intervertebral assembly according to the ninth embodiment of the current disclosure.

FIG. 20 is a cross sectional view of the assembled intervertebral assembly of the ninth embodiment of the current disclosure.

FIG. 21 is an exploded intervertebral assembly according to a tenth embodiment of the current disclosure.

FIG. 22 is an assembled intervertebral assembly according to the tenth embodiment of the current disclosure.

FIG. 23 is a cross sectional view of the assembled intervertebral assembly of the tenth embodiment of the current disclosure.

FIG. 24 is an exploded intervertebral assembly according to an eleventh embodiment of the current disclosure.

FIG. 25 is an assembled intervertebral assembly according to the eleventh embodiment of the current disclosure.

FIG. 26 is an exploded intervertebral assembly according to a twelfth embodiment of the current disclosure.

FIG. 27 is an exploded intervertebral assembly according to a twelfth embodiment of the current disclosure.

FIG. 28 is an assembled intervertebral assembly according to the twelfth embodiment of the current disclosure.

FIG. 29 is a cross-sectional view of the intervertebral assembly according to the twelfth embodiment of the current disclosure.

FIG. 30 is a cross-sectional view of the intervertebral assembly of the twelfth embodiment of the current disclosure in an articulated position.

### DETAILED DESCRIPTION

The present disclosure relates generally to the field of orthopedic surgery, and more particularly to an apparatus and method for vertebral reconstruction using a functional intervertebral prosthesis. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates.

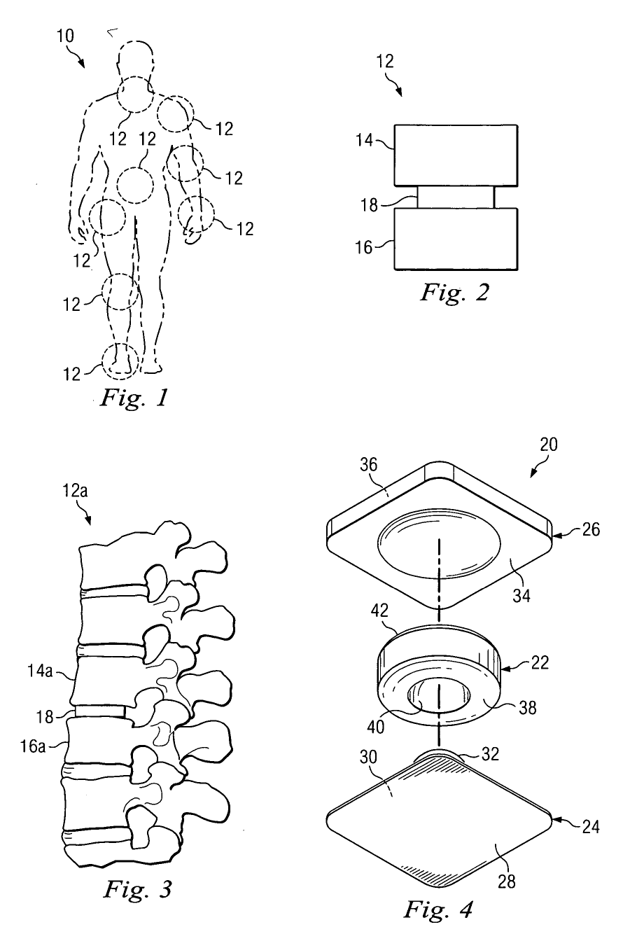

Referring first to FIG. 1, the numeral 10 refers to a human anatomy having one or more joint locations 12 which may be damaged by injury or disease. As shown in FIG. 2, in a typical arthroplasty procedure all or a portion of one of the joints 12 may be removed, creating a void between two intact bones 14, 16. An implant 18 may then be inserted between the bones 14, 16 to at least partially fill the void.

Referring now to FIG. 3, one example of a joint that can benefit from the present invention is a vertebral joint 12a with the implant 18 interposed between vertebrae 14a , 16a, corresponding to intact bones 14, 16, respectively. In a typical surgical discectomy, a void is created between the two intact vertebrae 14a and 16a. This procedure may be performed using an anterior, anterolateral, lateral, or other approach known to one skilled in the art. An implant 18 according to an embodiment of the present invention may then be provided to fill the void between the two intact vertebrae 14a and 16a.

Other examples of joints that can benefit from the present invention include orthopedic applications in shoulder, knee, or hip arthroplasty. It is understood that other joints may require different sizes, materials, and/or shapes to fulfill specific joint requirements, as is well understood by those of ordinary skill in the art. Sizing and material selection may, for example, require consideration of the heavy load bearing requirements of hip or knee joints. Other joints, such as cervical vertebrae joints, may require materials and sizing which reflect the wide range of movement desired at the joint.

The vertebral embodiments disclosed may be used in the cervical, thoracic, or lumbar spine or in other regions of the vertebral column. Although the embodiments to be described are generally premised upon the removal of a single disc, it is understood that more than one of the disclosed devices may be used in a multi-level disc replacement such as, for example, the replacement of two or more vertebral discs. The methods and apparatus of this disclosure may also be applied to the insertion of a vertebral body replacement device between two vertebrae following a corpectomy, in which at least one vertebral body has been removed. Moreover, the methods and apparatus may be used whenever motion preservation is needed or desired.

Referring now to FIG. 4, a joint prosthesis 20, which in this embodiment may be an intervertebral disc prosthesis, includes a center member 22 interposed between two endplate assemblies 24, 26. The endplate assembly 24 may include an exterior surface 28 and an interior surface 30. An articulation mechanism such as a protrusion 32 may extend from the interior surface 30. In this embodiment the protrusion may be semi spherical, however protrusions may be provided in a variety of shapes, a few of which will be described in other embodiments. The surfaces 28 and 30 may be flat, angled, or curved. In this embodiment, the exterior surface 28 may be relatively flat or may be contoured to match the surface of an adjacent vertebral endplate. The interior surface 30 may taper away from or toward the protrusion 32.

The endplate assembly 26 may include a interior surface 34 and an exterior surface 36. The surfaces 34 and 36 may be flat, angled, or curved. In this embodiment, the surface 36 may be generally flat or may be contoured to match the surface of an adjacent vertebral endplate. This surface may have other features (not shown), such as fins or keels, to secure the exterior surface 36 to the bone. The interior surface 34 may be generally concave and may serve as an articulation mechanism.

The center member 22 may vary somewhat in shape, size, composition, and physical properties, depending upon the particular joint for which the implant is intended or a particular deformity which the prosthesis 20 is intended to correct. The shape of the center member 22 may complement that of the interior surfaces 30, 34 of the endplate assemblies 24, 26 to allow for a range of translational, flexural, extensional, rotational, and lateral bending motion appropriate to the particular joint being replaced. In this embodiment, the center member 22 may include a surface 38 having a cavity 40 generally conforming to the shape of the protrusion 32. The center member 22 may also have a surface 42 which, in this embodiment, may generally conform to the shape of the interior surface 34.

The endplate assemblies 24, 26 and center member 22 may be formed of any suitable biocompatible material including, cobalt-chrome alloys, stainless steel, titanium alloys, alumina, zirconia, polycrystalline diamond, pyrolytic carbon, polyetheretherketone (PEEK), ultra-high molecular weight polyethylene (UHMWPE), cross-linked UHMWPE, and/or other suitable materials. The surfaces 28, 36 may include features or coatings which enhance the purchase of the implanted prosthesis. For example, a biocompatible and osteoconductive material such as hydroxyapatite (HA) may coat all or a portion of the surface 28. Other suitable coatings or treatments may include a porous bead coating, a porous mesh coating, osteogenic peptide coating, growth factor coating, rh-BMP coating, and/or grit blasting. Other suitable features may include serrations, spikes, ridges, fins, and/or other surface textures.

In some embodiments, the center member 22 may be formed of the relatively rigid materials listed above, and in other embodiments, the center member may permit a degree of elasticity or dampening, and accordingly, an elastomeric material may be used for the center member. Although the center member 22 may have a degree of flexibility, it may also be sufficiently stiff to effectively cooperate with the endplate assemblies to limit motion beyond an allowable range. The surface of the center member 22 may also be sufficiently durable to provide acceptable wear characteristics. In one embodiment, this combination of properties may be achieved with a center member 22 having surface regions that are harder than the material of the central body closer to its core. The portion 22 may, therefore, comprise a biocompatible composite or elastomeric material having a hardened surface.

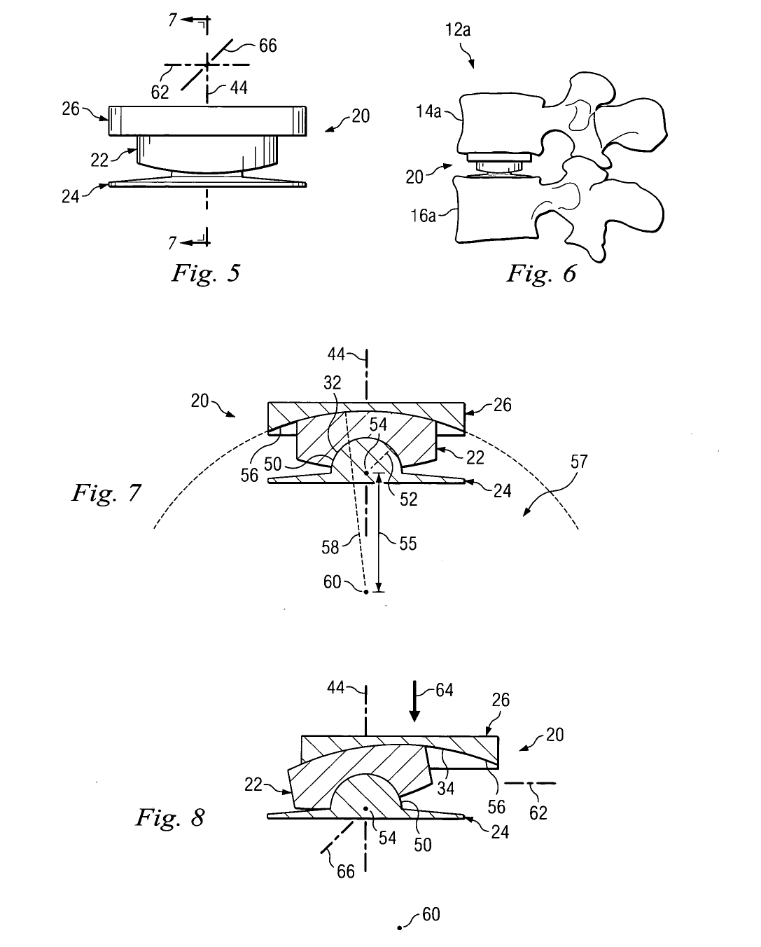

Referring now to FIG. 5, the components of the intervertebral disc prosthesis 20 may be assembled by engaging the protrusion 32 with the cavity 40 and by positioning the surface 42 of the center member on the surface 34 of the endplate assembly 26. The components 26, 22, 24 may be centrally aligned along a longitudinal axis 44.

Referring now to FIG. 6, the intervertebral disc prosthesis 20 may used as the implant 18 and may be inserted in the void of the vertebral column 12a (of FIG. 3) created by the discectomy. In one embodiment, the surface 36 may contact an endplate of vertebra 14a and the surface 28 may contact the endplate of vertebra 16a. In other embodiments, the prosthesis may be inverted.

As shown in the cross sectional view of FIG. 7, the intervertebral disc prosthesis 20 may be in a neutral position when the components 26, 22, 24 are centrally aligned along the longitudinal axis 44. The protrusion 32 may have a curve 50, which in this embodiment may be an arc with a relatively constant radius 52 and a center point 54. The surface 34 may have a curve 56 which in this embodiment may be an arc with a relatively constant radius 58 and a center point 60. A distance 55 may be measured between the center points 54, 60. In this example, the radius 52 is smaller than the radius 58, and accordingly, the arc 50 is tighter than the arc 56. In the neutral position, the center points 54 and 60 may be aligned along the longitudinal axis 44, and the smaller curve 50 may be positioned within the curve 56, which in this embodiment may be the area 57 defined by the sweep of the radius 58.

FIG. 8 shows the intervertebral disc prosthesis 20 in a translated position along, for example, an anterior-posterior axis 62. Translation may, for example, occur with flexion-extension movement. As the endplate assemblies 24, 26 are moved out of alignment relative to axis 44, the center member 22 may articulate between the endplate assembly interior surfaces 30, 34. With the patient's body weight as a load 64 in the longitudinal direction 44 and the position of the smaller curve 50 within the larger curve 56, the prosthesis 20 may be biased to return to the more stable, neutral position in which the curves 50, 56 are aligned along the longitudinal axis 44. In this embodiment alignment may occur when the center points 54, 60 are aligned along the longitudinal axis 44. In this embodiment alignment may occur when the center points 54, 60 are aligned along the longitudinal axis 44. This embodiment describes curves which represent arcs of circle, but in alternative embodiments the curves may be portions of other curves, such as an arc of an ellipse. In these alternative embodiments, alignment may occur when foci, for example of an ellipse, are in alignment or when center lines bisecting the curves are in alignment.

This tendency of the prosthesis 20 to self correct a spondylolisthesis or other displacement may allow freer, more natural joint movement while preventing excessive translation that could otherwise result in instability of the prosthesis 20. Instability may result in the placement of unsustainable loads on adjacent joints or may result in the disassembly of the prosthesis 20. The alignment bias of the prosthesis 20 may relieve excessive loads that might otherwise form in adjacent joints due to chronic over-displacement of the endplate assemblies 24, 26. Although the wider arc is superior to the tighter arc in the orientation of this embodiment, in another embodiment, the orientation may be inverted with the tighter arc superior to the wider arc but with the tighter arc still falling within the curve of the wider arc.

It may be appreciated that the amount of alignment bias, and accordingly the amount of stability, may be related to the distance 55 between the center points 54, 60. As the distance 55 increases (for example, a sphere on a flat surface), stability, the amount of constraint within the prosthesis 20, and the tendency to self-align may decrease. As the distance 55 decreases (for example, a sphere in a tight socket), stability, constraint within the prosthesis 20, and the tendency to self-align may increase. Although this embodiment has been described as contemplating a displacement in the anterior-posterior direction 62, displacements caused by translation, bending, and/or rotation in other directions or combinations of directions may be corrected using other embodiments of the invention. For example, displacement of the endplate assembly 26 relative to the endplate 24 in a lateral direction 66 may also generate constraining forces which drive the center points 54, 60 back into alignment. The components 22-26 may be selected from a kit which allows the surgeon to design a patient specific prosthesis having a patient-appropriate amount of constraint and bias.

In embodiments involving multi-level disc removal, ligaments and other supportive soft tissue structures may be surgically removed or compromised. In these embodiments, replacing the discs with assemblies, such as prostheses 20, may resupply at least some of the stability lost with the removal of the soft tissue. This restored stability may prevent excessive loading and wear in the adjacent joints and may also encourage more kinematically accurate motions.

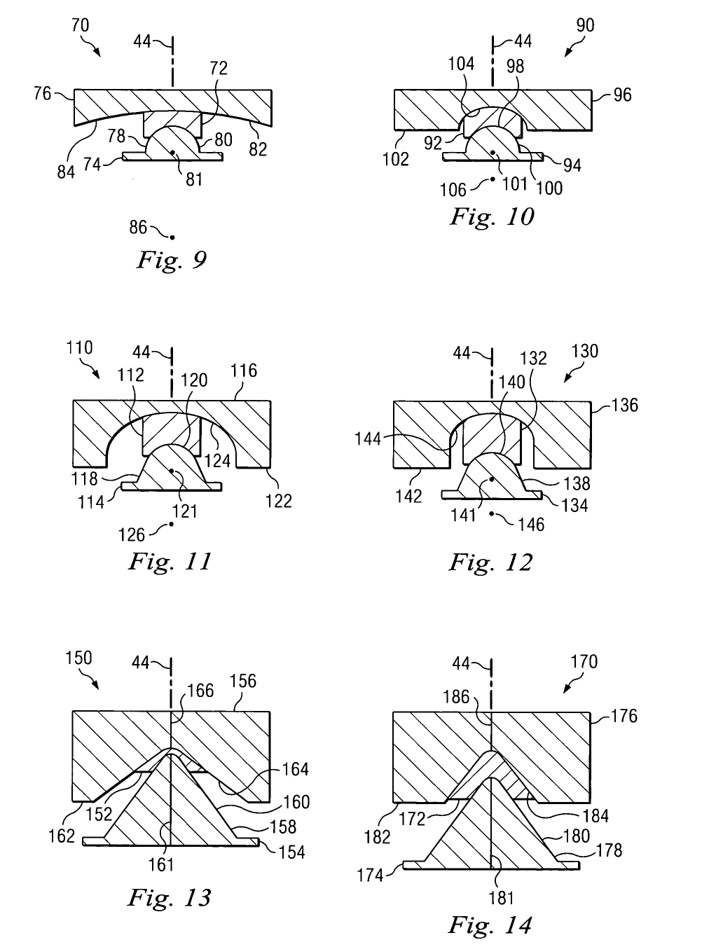

Referring now to FIG. 9, in this embodiment, an intervertebral disc prosthesis 70, may include a center member 72 interposed between two endplate assemblies 74, 76. The endplate assembly 74 may include a protrusion 78 having a curve 80. In this embodiment, the curve 80 may be an arc having a centerpoint 81 and a constant radius. The endplate assembly 76 may include an interior surface 82 which may have a curve 84. In this embodiment, the curve 84 may be an arc having a center point 86 and a constant radius.

Referring now to FIG. 10, in this embodiment, an intervertebral disc prosthesis 90, may include a center member 92 interposed between two endplate assemblies 94, 96. The endplate assembly 94 may include a protrusion 98 having a curve 100. In this embodiment, the curve 100 may be an arc having a center point 101 and a constant radius. The endplate assembly 96 may include an interior surface 102 which may have a curve 104. In this embodiment, the curve 104 may be an arc having a center point 106 and a constant radius.

The materials, the assembly, and the operation of prosthesis 90 may be similar to prosthesis 20 and therefore will not be described in detail. The shape of a protrusions relative to the shape of the contacted interior surfaces may correspond to the amount of constraint within the prosthesis. For example, where the arc-shaped curve 84 is wide compared to the relatively tight curve 104 in FIG. 9, the prosthesis 70 may be more constrained than prosthesis 90 in the embodiment of FIG. 10 wherein the arc-shaped curve 104 more closely matches the curve 100. Increased constraint may correspond to an increased bias for the prosthesis to return to the neutral position with the center points centrally aligned about the longitudinal axis 44.

Referring now to FIG. 11, in this embodiment, an intervertebral disc prosthesis 110, may include a center member 112 interposed between two endplate assemblies 114, 116. The endplate assembly 114 may include a protrusion 118 having a curve 120. In this embodiment, the curve 120 may be a semi-ellipse or other type of curve having a focus point 121 and a variable radius. The endplate assembly 116 may include an interior surface 122 which may have a curve 124. In this embodiment, the curve 124 may be U-shaped having a focus point 126, a variable radius, angled flat, and/or parallel flat portions. The materials and the assembly of prosthesis 110 may be similar to prosthesis 20 and therefore will not be described in detail. In operation, the prosthesis 110 may be biased toward alignment of the foci 121, 126 about the longitudinal axis 44.

Referring now to FIG. 12, in this embodiment, an intervertebral disc prosthesis 130, may include a center member 132 interposed between two endplate assemblies 134, 136. The endplate assembly 134 may include a protrusion 138 having a curve 140. In this embodiment, the curve 140 may be a semi-ellipse having a focus point 141 and a variable radius. The endplate assembly 136 may include an interior surface 142 which may have a curve 144. In this embodiment, the curve 144 may be U shaped having a focus point 146, a variable radius, angled flat, and/or parallel flat sections.

The materials and the assembly of prostheses 110, 130 may be similar to prosthesis 20 and therefore will not be described in detail. In operation, the prosthesis 130 may be biased toward alignment of the foci 141, 146 about the longitudinal axis 44. As shown in FIGS. 11 and 12, in some embodiments, the shape of the curves 124, 144 may not correspond to constant radius arcs of a circle, but rather the shape of the curve may be, for example, a U-shape, a semi-ellipse, or an elliptic curve. In FIG. 11 where the U-shaped curve 124 is wide compared to the relatively tight curve 144 of FIG. 12, the prosthesis 110 may be less constrained than prosthesis 130 wherein the U-shaped curve 154 is relatively tight and more closely matches the curve 140. It may be appreciated that the prosthesis 110 (FIG. 11) may be more constrained than prosthesis 70 (FIG. 9) as the walls of the U-shape may increase the bias for the prosthesis 110 to return to the neutral position.

Referring now to FIG. 13, in this embodiment, an intervertebral disc prosthesis 150, may include an center member 152 interposed between two endplate assemblies 154, 156. The endplate assembly 154 may include a protrusion 158 having a curve 160. In this embodiment, the curve 160 may have a combination of curved and flat surfaces and may have a center line 161 bisecting the curve 160. The endplate assembly 156 may include an interior surface 162 which may have a curve 164. In this embodiment, the curve 164 may have a combination of curved and flat surfaces and may have a center line 166 bisecting the curve 164. The materials and the assembly of prosthesis 150 may be similar to prosthesis 20 and therefore will not be described in detail. In operation, the prosthesis 150 may be biased toward alignment of the center lines 161, 166 along the axis 44.

Referring now to FIG. 14, in this embodiment, an intervertebral disc prosthesis 170, may include an center member 172 interposed between two endplate assemblies 174, 176. The endplate assembly 174 may include a protrusion 178 having a curve 180. In this embodiment, the curve 180 may have a combination of curved and flat surfaces and may have a center line 181 bisecting the curve 180. The endplate assembly 176 may include an interior surface 182 which may have a curve 184. In this embodiment, the curve 184 may have a combination of curved and flat surfaces and may have a center line 186 bisecting the curve 180. The materials, the assembly, and the operation of prosthesis 170 may be similar to prosthesis 20 and therefore will not be described in detail.

For prostheses 150, 170, the curves 164, 184 are relatively pointed compared to curve 80 (FIG. 9). In FIG. 13 where the pointed curve 164 is wide compared to the relatively tight curve 184 of FIG. 12, the prosthesis 150 may be less constrained than prosthesis 170 wherein the U-shaped curve 184 is relatively tight and more closely matches the curve 180.

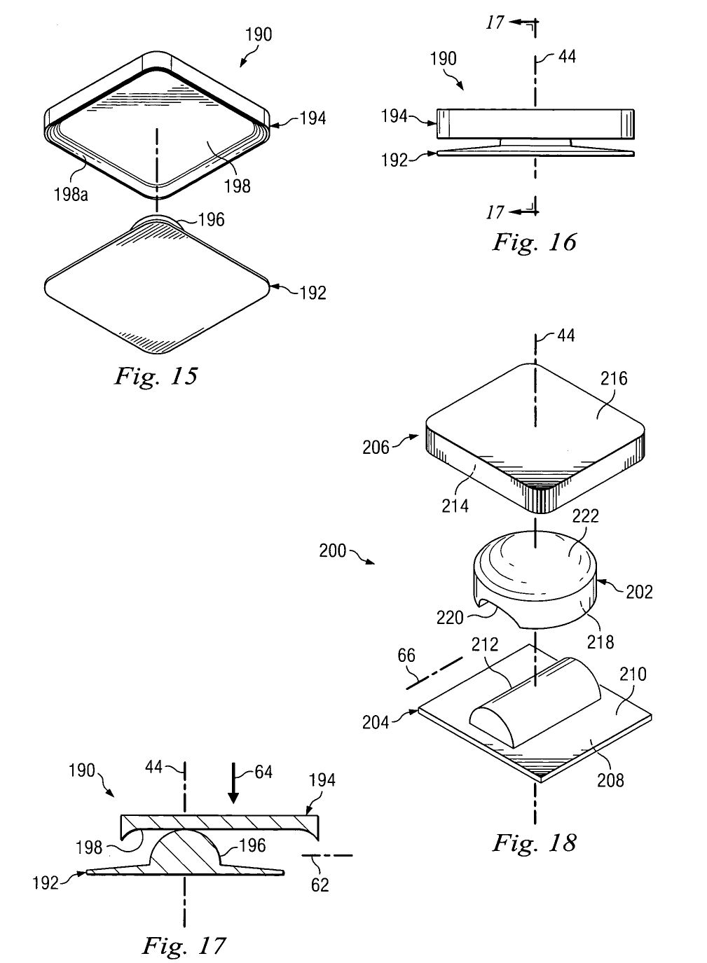

Referring now to FIG. 15, an intervertebral disc prosthesis 190 may include two endplate assemblies 192, 194 which may be identical or substantially similar to endplate assemblies 24, 26 (FIG. 4) and therefore, will not be described in detail except to define a protrusion 196 corresponding to protrusion 32 of prosthesis 20, and a surface 198 corresponding to surface 34. As shown in FIG. 16, the prosthesis 190 may be assembled by positioning the protrusion 196 on the surface 198. The components, 192, 194 may be aligned along the longitudinal axis 62. The prosthesis 190 of this embodiment is one example of a relatively unconstrained joint (as compared to FIG. 10, for example). Protrusion 196 may be permitted to move unconstrained on surface 198 as the patient moves. The surface 198 may, in some embodiments as shown, have a slight lip 198a around the perimeter to provide a minimal amount of constraint. FIG. 17 shows the intervertebral disc prosthesis 190 in a translated position along, for example, an anterior-posterior axis 62. This embodiment, which may omit a bushing, center articulating portion, or other wear reduction device, may be suitable, for example, when contacting surfaces are formed of extremely durable material able to withstand point contact. This embodiment may also minimize stress on the adjacent vertebral endplates.

Referring now to FIG. 18, a joint prosthesis 200, which in this embodiment may be an intervertebral disc prosthesis, includes a center member 202 interposed between two endplate assemblies 204, 206. The endplate assembly 204 may include an exterior surface 208 and an interior surface 210. A protrusion 212 may extend from the interior surface 210. In this embodiment, the protrusion 212 may be a semi-cylinder extended in the direction of axis 66, however, as described above, protrusions may be provided in a variety of shapes suitable for a particular application or particular location in the vertebral column. The surfaces 208 and 210 may be flat, angled, or curved. In this embodiment, the exterior surface 208 may be relatively flat or may be contoured to match the surface of an adjacent vertebral endplate. The interior surface 210 may taper away from the protrusion 212.

The endplate assembly 206 may include a interior surface 214 and an exterior surface 216. The surfaces 214 and 216 may be flat, angled, or curved. In this embodiment, the surface 216 may be generally flat or may be contoured to match the surface of an adjacent vertebral endplate. The interior surface 214 may be generally concave.

The center member 202 may vary somewhat in shape, size, composition, and physical properties, depending upon the particular joint for which the implant is intended. The shape of the center member 202 may complement that of the interior surfaces 210, 214 of the endplate assemblies 204, 206, respectively, to allow for a range of translational, flexural, extensional, rotational, and lateral bending motion appropriate to the particular joint being replaced. In this embodiment, the center member 202 may include a surface 218 having a cavity 220 generally conforming to the shape of the protrusion 212. The center member 202 may also have a surface 222 which, in this embodiment, may generally conform to the shape of the interior surface 214.

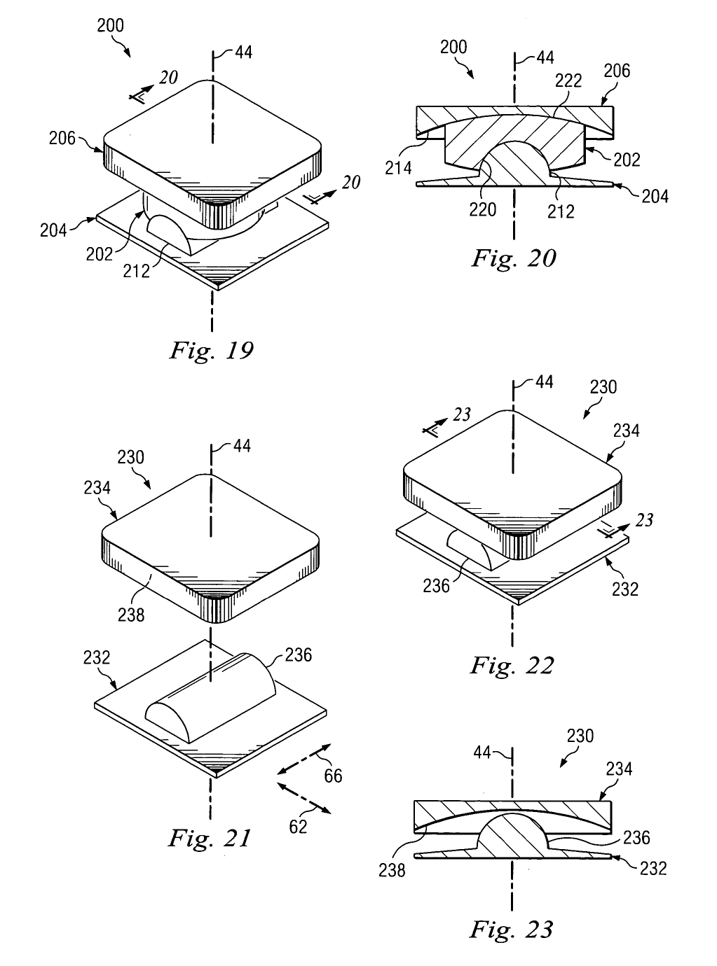

The components 202, 204, 206 may be formed from the same materials as described above for components 22, 24, 26, respectively. Referring now to FIGS. 19 & 20, the components of the intervertebral disc prosthesis 200 may be assembled by engaging the protrusion 212 with the cavity 220 and positioning the surface 222 of the center member 202 on the surface 214. The components 202-206 may be centrally aligned along the longitudinal axis 44. The intervertebral disc prosthesis 200 may be inserted in the void of the vertebral column 12a (of FIG. 3) created by discectomy. The positioning and functioning of the prosthesis 200 may be similar to that of the prosthesis 20 and therefore will not be described in detail. As described above for prosthesis 20, the prosthesis 200 may also have a bias to return toward a neutral position centrally aligned along the axis 44. Additionally, in this embodiment, the extension of the protrusion 212 in the lateral direction 66 may permit more stable and controlled lateral translation while decreasing the risk of dislodging the center member 202.

Referring now to FIG. 21, an intervertebral disc prosthesis 230 may include two endplate assemblies 232, 234 which may be identical or substantially similar to endplate assemblies 204, 206 (FIG. 18-20) and therefore, will not be described in detail except to define a protrusion 236 similar to protrusion 212 of prosthesis 200, and a surface 238 similar to surface 214. As shown in FIGS. 22 and 23, the prosthesis 230 may be assembled by positioning the protrusion 236 on the surface 238. The components 232, 234 may be centrally aligned along the longitudinal axis 44. The curved surface 238 and the curve of the protrusion 236 may provide constraint in the direction 62, but may provide relatively little constraint in direction 66. As shown, the protrusion may be relatively linear along the axis 66, but in other examples, the protrusion may be curved along the axis 66 to create an elliptical dome which provides constraint in both directions 62, 66. Prosthesis 230, which may omit a bushing, center articulating portion, or other wear reduction device, may be suitable, for example, when contacting surfaces are formed of extremely durable material able to withstand line contact.

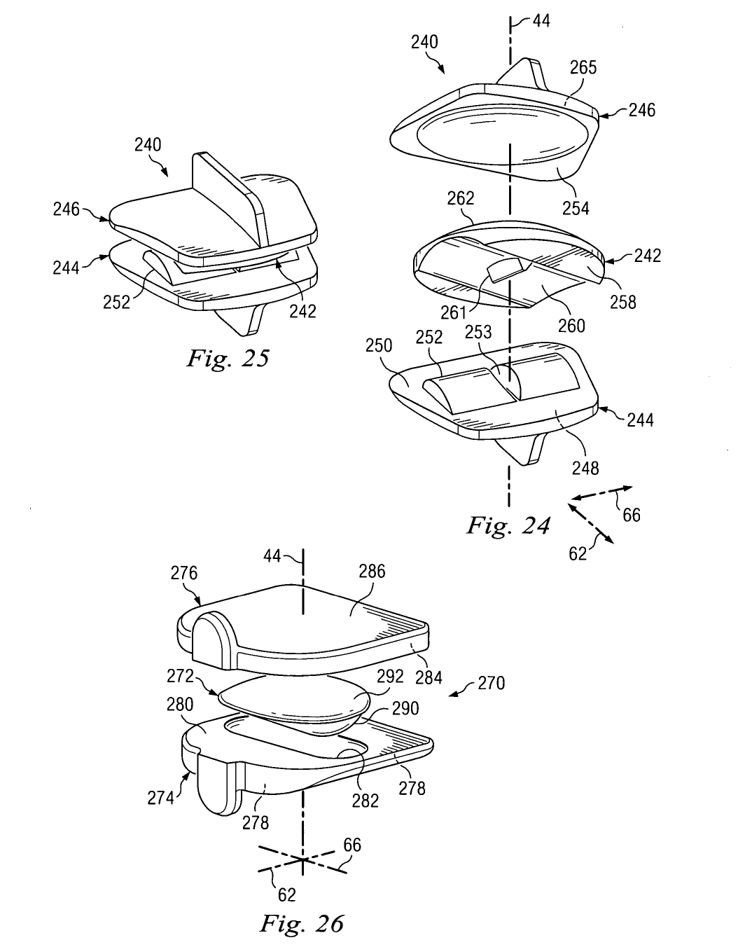

Referring now to FIG. 24, a joint prosthesis 240, which in this embodiment may be an intervertebral disc prosthesis, includes a center member 242 interposed between two endplate assemblies 244, 246. The endplate assembly 244 may include an exterior surface 248 and an interior surface 250. A protrusion 252 may extend from the interior surface 250. In this embodiment, the protrusion 252 may be a semi-cylinder extended along the direction of axis 66. A restraint member 253, which in this example may be a depression, may be formed on the protrusion 252 or the surface 250. The restraint member 253 may extend across the protrusion 252 in the anterior-posterior direction 62 and may be flared to permit limited motion in the lateral direction 66. The surfaces 248 and 250 may be flat, angled, or curved. In this embodiment, the exterior surface 248 may be relatively flat or may be contoured to match the surface of an adjacent vertebral endplate. The interior surface 250 may taper away from the protrusion 252.

The endplate assembly 246 may include a interior surface 254 and an exterior surface 256. The surfaces 254 and 256 may be flat, angled, or curved. In this embodiment, the surface 256 may be generally flat or may be contoured to match the surface of an adjacent vertebral endplate. The interior surface 254 may be generally concave.

The center member 242 may vary somewhat in shape, size, composition, and physical properties, depending upon the particular joint for which the implant is intended. The shape of the center member 242 may complement that of the interior surfaces 250, 254 of the endplate assemblies 244, 246, respectively, to allow for a range of translational, flexural, extensional, rotational, and lateral bending motion appropriate to the particular joint being replaced. In this embodiment, the center member 242 may include a surface 258 having a cavity 260 generally conforming to the shape of the protrusion 252. The cavity 260 may comprise a restraint mechanism 261 which, in this example, may be a boss. More than one restraint mechanism 261 may be used (corresponding to more than one restraint mechanism 253), and the one or more restraint mechanisms 261 may be located at alternative locations on center member 242. The boss 261 may extend across the cavity 260 in the anterior-posterior direction 62 to restrict motion along the axis 66, but in other examples a restraint mechanism may be positioned to restrict motion along the axis 62. The center member 242 may also have a surface 262 which, in this embodiment, may generally conform to the shape of the interior surface 254.

The components 242, 244, 246 may be formed from the same materials as described above for components 22, 24, 26, respectively. Referring now to FIG. 25, the components of the intervertebral disc prosthesis 240 may be assembled by engaging the protrusion 252 with the cavity 260 and further engaging the restraint mechanism 261 with the restraint member 253. The surface 262 of the center member 242 may be positioned on the surface 254. The components 242-246 may be centrally aligned along the longitudinal axis 44.

The intervertebral disc prosthesis 240 may be inserted in the void of the vertebral column 12a (of FIG. 3) created by the removal of disc 12. The positioning and functioning of the prosthesis 240 may be similar to that of the prosthesis 200 (FIG. 18) and therefore will not be described in detail. As described above in detail for prostheses 20 and 200, the prosthesis 240 may have a bias to return toward the neutral position aligned along the axis 44. Additionally, in this embodiment, the extension of the protrusion 252 in the lateral direction 66 may permit more stable and controlled lateral translation while decreasing the risk of dislodging the center member 242. The engagement of the restraint mechanism 261 and the restraint member 253 may limit lateral translation in accordance with the needs of a particular application. The lateral flare of the restraint member 253 may be varied such that embodiments having a narrow flare would permit less lateral translation than embodiments having wider flares. It is understood that a variety of other restraint mechanism 261/restraint member 253 configurations may be employed to restrict the amount of lateral translation. For example, the restraint member 253 can protrude to engage a grooved restraint mechanism 261.

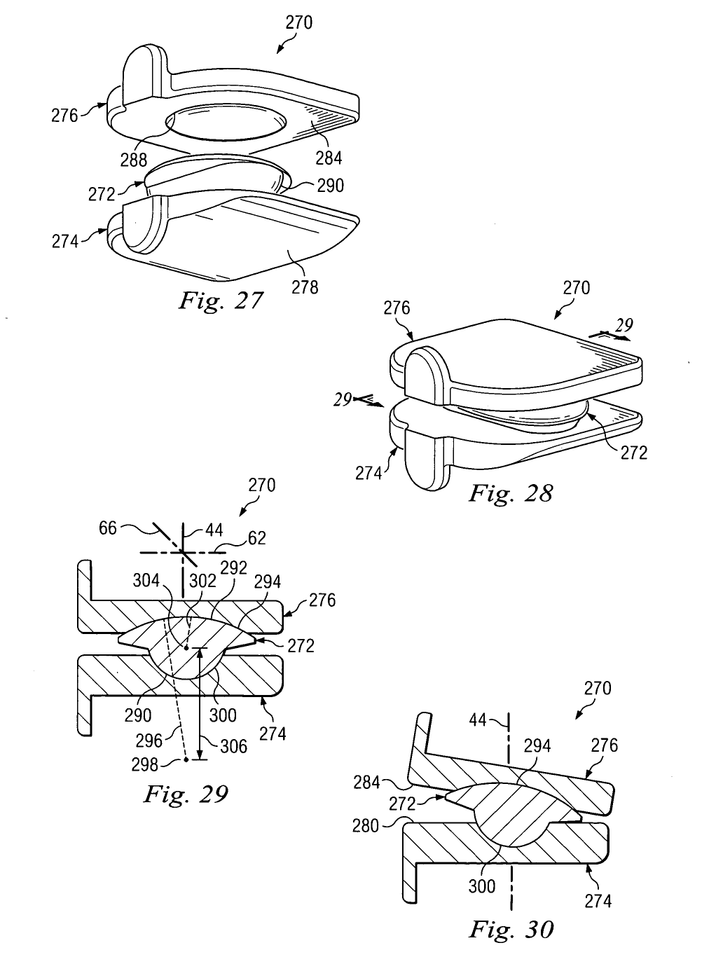

Referring now to FIGS. 26-30, a joint prosthesis 270, which in this embodiment may be an intervertebral disc prosthesis, includes a center member 272 interposed between two endplate assemblies 274, 276. The endplate assembly 274 may include an exterior surface 278 and an interior surface 280. A depression 282, may be formed on the interior surface 280. In this embodiment, the depression 282 may be formed as a concave recess extended along the lateral direction of axis 66. The depression 282 may also be curved along the axis 66. The surfaces 278 and 280 may be flat, angled, or curved. In this embodiment, the exterior surface 278 may be relatively flat or may be contoured to match the surface of an adjacent vertebral endplate. The interior surface 280 may be generally flat around the depression 282.

The endplate assembly 276 may include a interior surface 284 and an exterior surface 286. The surfaces 284 and 286 may be flat, angled, or curved. In this embodiment, the surface 286 may be generally flat or may be contoured to match the surface of an adjacent vertebral endplate. The interior surface 284 may include a concave recess 288.

The center member 272 may vary somewhat in shape, size, composition, and physical properties, depending upon the particular joint for which the implant is intended. The shape of the center member 272 may complement that of the interior surfaces 280, 284 of the endplate assemblies 274, 276, respectively, to allow for a range of translational, flexural, extensional, rotational, and lateral bending motion appropriate to the particular joint being replaced. In this embodiment, the center member 272 may include a surface 290 generally conforming to the shape of the depression 282. The center member 272 may also have a surface 292 which, in this embodiment, may generally conform to the shape of the concave recess 288.

As shown in FIG. 29, the intervertebral disc prosthesis 270 may be in a neutral position when the components 272-276 are centrally aligned along the longitudinal axis 44. The surface 292 may have an arc 294 with a radius 296 and a center point 298. The surface 290 may have an arc 300 with a radius 302 and a center point 304. In the neutral position of FIG. 29, the center points 298, 304 are aligned along the longitudinal axis 44. In this example, the radius 302 is smaller than the radius 296, and accordingly, the arc 300 is tighter than the arc 294. A distance 306 extends between the center points 298, 304.

The components 272, 274, 276 may be formed from the same materials as described above for components 22, 24, 26, respectively. Referring specifically to FIG. 28-30, the components of the intervertebral disc prosthesis 270 may be assembled by engaging the surface 290 with the depression 282 and further engaging the surface 292 with the surface 288. The components 272-276 may be centrally aligned along the longitudinal axis 44. The intervertebral disc prosthesis 270 may be inserted in the void of the vertebral column 12a (of FIG. 3) created by the removal of disc 12. The surface 278 may contact an endplate of vertebra 16 and the surface 286 may contact the endplate of vertebra 14a.

Referring now to FIG. 30, the intervertebral disc prosthesis 270 may be articulated by, for example, flexion, extension, and/or translational movement. In response to this movement, the center member 272 may articulate between the endplate assembly interior surfaces 284, 280. With the position of the tighter arc 300 within the wider arc 294, the articulated prosthesis 270 may be constrained and biased to return to the more stable, neutral position aligned along the longitudinal axis 44 when subject to a load such as the patient's weight. This tendency of the prosthesis 270 to self align may allow more natural joint movement while preventing excessive translation that might otherwise result in the disassembly of the prosthesis 270. Further, this alignment bias may relieve excessive loads that might otherwise form in adjacent joints due to chronic over-displacement between the center points 298, 304. The depression 282 and the concave recess 288, in addition to permitting the smooth articulation of the center member 272, may function to limit or prohibit lateral movement along the axis 66. The matching curvatures of surfaces 282,290 and 292,288 may distribute the loadings and enhance the wear resistance of the components 272, 274, 276. The components 272, 274, 276 may be modular which may permit the selection of a center member 272 having a thickness which adjusts the prosthesis 270 to a desired height.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. In the claims, means-plus-function clauses are intended to cover the structures described herein as performing the recited function and not only structural equivalents, but also equivalent structures.

1. A joint prosthesis comprising:
a first member for engaging a first bone portion, the first member comprising a first surface with a first curve;
a second member for engaging a second bone portion, the second member comprising a second surface with a second curve;
wherein the first member is translatable with respect to the second member and the second curve is positioned within the first curve to bias the first and second curves towards alignment along a first axis passing through the first and second bone portions.

2. The joint prosthesis of claim 1 wherein the first curve has a first constant radius and a first center point, and the second curve has a second constant radius and a second center point.

3. The joint prosthesis of claim 2 wherein the first constant radius is larger than the second constant radius.

4. The joint prosthesis of claim 2 wherein alignment comprises alignment of the first and second center points along the first axis.

5. The joint prosthesis of claim 2 wherein the first curve has a first interior area defined by the sweep of the first constant radius and the second curve is positioned within the interior area.

6. The joint prosthesis of claim 1 wherein the first curve has a variable radius.

7. The joint prosthesis of claim 1 wherein the first curve has a combination of curved and flat portions.

8. The joint prosthesis of claim 1 further comprising a center member interposed between the first and second members.

9. The joint prosthesis of claim 8 wherein the center member articulates between the first and second surfaces as the first member is translated relative to the second member.

10. The joint prosthesis of claim 1 wherein the second surface has a semi-cylindrial protrusion extended along a lateral axis.

11. The joint prosthesis of claim 1 wherein the second surface has a semi-spherical protrusion.

12. The joint prosthesis of claim 1 wherein the first and second surfaces have depressions.

13. The joint prosthesis of claim 1 further comprising a restraint mechanism for restricting motion along a second axis orthogonal to the first axis.

14. The joint prosthesis of claim 1 wherein the first member is translatable with respect to the second member along a third axis orthogonal to the first and second axes.

15. The joint prosthesis of claim 1 further comprising a neutral position and a first position wherein in the first position, the implant is biased to move toward the neutral position.

16. The joint prosthesis of claim 15 wherein in the first position, the first curve is in closer conformance with the second curve.

17. The joint prosthesis of claim 1 wherein the first curve is wider than the second curve.

18. The joint prosthesis of claim 1 wherein the first curve is superior to the second curve along the first axis.

19. The joint prosthesis of claim 1 wherein the first surface is concave and the second surface is convex.

20. The joint prosthesis of claim 1 wherein the first and second surfaces are concave.

21. The joint prosthesis of claim 1 wherein the first and second bone portions comprise a shoulder joint.

22. The joint prosthesis of claim 1 wherein the first and second bone portions comprise a knee joint.

23. The joint prosthesis of claim 1 wherein the first and second bone portions comprise a hip joint.

24. A joint prosthesis comprising:
a first member for engaging a first bone portion, the first member comprising a first curved surface;
a second member for engaging a second bone portion, the second member comprising a second curved surface;
wherein as the first member is translated with respect to the second member, conformity between the first and second curved surfaces increases.

25. A method for installing a joint prosthesis device between two bone portions, the method comprising:
engaging a center member with a first curved surface of a first member;
engaging the center member with a second curved surface of a second member;
positioning the second curved surface within an interior area of the first curved surface;
engaging the first member with a first bone portion; and
engaging the second member with a second bone portion,
wherein the first member is translatable and further wherein the first and second curved surfaces are biased toward alignment along an axis passing through the first and second bone portions.

26. A joint prosthesis comprising:
a first member for engaging a first bone portion, the first member comprising a first relatively flat surface, wherein the first relatively flat surface includes a perimeter lip;
a second member for engaging a second bone portion, the second member comprising a second curved surface;
wherein the first member is translatable with respect to the second member and wherein the second curve is positioned on the first relatively flat surface, within the perimeter lip allowing the second member to move unconstrained within perimeter lip.
